(19) United States
(12) Patent Application Publication
(10) Pub. No.: US 2016/0110519 A1
(43) Pub. Date: Apr. 21, 2016

(54) MEDICATION UNIQUE DRUG IDENTIFIER TRACKING, MONITORING AND VERIFICATION FOR PHARMACY

(71) Applicant: GSL Solutions, Inc., Vancouver, WA (US)

(72) Inventors: Shelton Louie, Vancouver, WA (US); Joseph Intile, Tualatin, OR (US); Stephen A. Garrett, Vancouver, WA (US)

(21) Appl. No.: 14/885,960

(22) Filed: Oct. 16, 2015

Related U.S. Application Data
(60) Provisional application No. 62/064,911, filed on Oct. 16, 2014.

(51) Int. Cl. G06F 19/00

(52) U.S. Cl. CPC G06F 19/3456

(57) ABSTRACT

An automated tracking system for a pharmacy that maintains each medication's Unique Drug Identifier (“UDI”) information from the stock supply container to individual prescriptions filled therefrom. The UDI information automatically travels with each individual prescription until it is dispensed to a customer or patient, thereby allowing the medication contained therein to be returned to stock without risk of losing key information about the medication such as its expiration date and the like. In disclosed embodiments, the computer system can monitor key UDI information of the stock supply containers and the individual filled prescriptions to promote use of medications expiring sooner than others in the pharmacy, locate, track and promote use of returned to stock medications that have not been returned to the supply containers, and prevent the dispensing of expired or about to expire medications.

### CROSS-REFERENCE TO RELATED APPLICATION

This application claims priority to U.S. provisional patent application Ser. No. 62/064/911 filed on Oct. 16, 2014, the disclosure of which is hereby incorporated by reference.

### BACKGROUND

Automated systems and methods for monitoring and tracking prescription orders, pharmacy supply containers, filled prescriptions and the like are known. Examples of these systems can be found in U.S. Pat. No. 8,479,988 to Louie, et al., and U.S. Pat. No. 7,747,477 to Louie et al., the disclosures of which are hereby incorporated by reference.

Also, local retail pharmacies, healthcare facilities and the like are increasingly relying on remote, “central fill” pharmacies to fill prescription orders of customers and patients and ship the filled prescriptions to the local retail pharmacy or healthcare facility for dispensing to the customer or patient. An exemplar, remote “central fill” pharmacy system is shown in U.S. Pat. No. 7,448,544 to Louie et al., the disclosure of which are hereby incorporated by reference.

In addition, automated prescription filling machines are known and in common use. These machines automatically count and dispense a desired medication into a container and apply a label containing customer identifying information. Some of these machines include automated verification systems, such as measuring the weight of the filled prescription and comparing it to a predetermined weight and flagging an error if the detected weight differs from the predetermined weight. These systems can also include automated visual detection systems such as cameras or the like that can physically count the number of pills dispensed and the appearance of the pills and compare those detected values with predetermined criteria and flag any discrepancies detected. Exemplar automated filling system are shown in U.S. Pat. No. 8,275.481 to Rice et al., U.S. patent application Ser. No. 13/473,287 to Luciano, et a., and U.S. patent application Ser. No. 13/325,782 to Rhoads, the disclosures of which are hereby incorporated by reference. These automated verification systems have proven themselves to be accurate and reliable.

In general, a licensed pharmacy technician or pharmacist is required to verify that a prescription for a customer has been properly filled. This verification usually includes verifying that the proper medication has been loaded into the correct locations within an automated filling machine, and that the proper amount of that medication has been placed in the container earmarked for the customer/patient, and that the customer's/patient's container is properly labeled. Labeling includes identifying information about the medication, quantity, how to take the medication contained therein and the customer/patient it is intended for.

In cases where a prescription order is filled at a remote facility, there may be several licensed pharmacy technicians and/or pharmacists along the filling flow process that perform the same verification steps for the same prescription and supply container orders. While such redundant verification is certainly beneficial in the rare case where verification along the flow process is erroneous, it needlessly increases the workload of the pharmacy technicians/pharmacists. Moreover, it requires more licensed pharmacy technicians & pharmacists to be involved in the fling and dispensing process than needed to accurately and consistently dispense the correct medications to patients and customers. Since the demand on licensed pharmacy technicians and pharmacists is continuing to increase, these redundant verification systems can compromise a pharmacy's ability to timely dispense medications to patients and customers.

Moreover, most medication has Unique Drug identity (“UDI”) information associated with it including its lot number, expiration date, pedigree code, National Drug Code (“NDC”), Drug Identification Number (“DIN”) and the like. This information is provided by the manufacturer of the medication with the supply container. However, much of this information is lost for medication withdrawn from the supply containers.

### SUMMARY

Thus, despite the known systems for filling prescription orders of customers and patients and filling and loading supply containers containing medications into automated filling machines, there remains a need for a cost effective system that consistently and accurately verifies prescription orders and the contents of supply containers without needlessly requiring multiple verifications from different licensed pharmacy technicians or pharmacists.

Moreover, there remains a need for medication UDI information of a supply of medication to follow the filling flow through a system to each individual filled prescription order of a customer or patient. The present invention fulfills these and other needs.

The advantages and features of novelty characterizing aspects of the invention are pointed out with particularity in the appended claims. To gain an improved understanding of the advantages and features of novelty, however, reference may be made to the following descriptive matter and accompanying figures that describe and illustrate various configurations and concepts related to the invention.

### FIGURE DESCRIPTIONS

The foregoing Summary and the following Detailed Description will be better understood when read in conjunction with the accompanying figures.

FIG. 1 is flow chart of an exemplar pharmacy filling system that uses a central fill pharmacy to fill the prescription order and a local pharmacy to dispense the prescription order to a customer or patient in accordance with an embodiment of the present invention. The automated filling machine is filled with medications contained in bins that have been filled and verified by a pharmacist or licensed pharmacy technician at a third party location, such as a wholesaler, a pharmaceutical manufacturer, or a central fill location.

FIG. 2 is a flow chart of an exemplar pharmacy filling system that uses a local pharmacy or healthcare facility to fill the prescription orders by relying on a third party, such as a wholesaler or pharmaceutical manufacturer, to provide bins filled with supply medications that Have been filed and verified by a pharmacist or licensed pharmacy technician at the third party location.

FIG. 3 is a schematic diagram of an exemplar filing of an individual prescription order of a customer from a stock supply of the prescribed medication showing the transfer from Unique Drug Identifier (“UDI”) information about the medication in the stock supply container to a computer readable tag that travels with the individual prescription order in accordance with an embodiment of the present invention. In a disclosed embodiment a computer system uses the tag to track, monitor and locate the individual prescription, correlate it with a customer, and maintain UDI information of the medication contained with the individual prescription.

FIG. 4 is a schematic diagram of an exemplar “Return to Stock” comparison whereby the computer system compares the UDI of an individual prescription order that was not timely dispensed to a customer or patient to the UDI of a supply container and authorizes the return of the medication to the stock supply container only if the UDI's between them are identical.

FIG. 5 is a flow chart of an exemplar prescription order filling system with a computer system monitored and verified “return to stock” feature that minimizes medication loss while preserving UDI integrity of the returned to stock medications.

### DETAILED DESCRIPTION

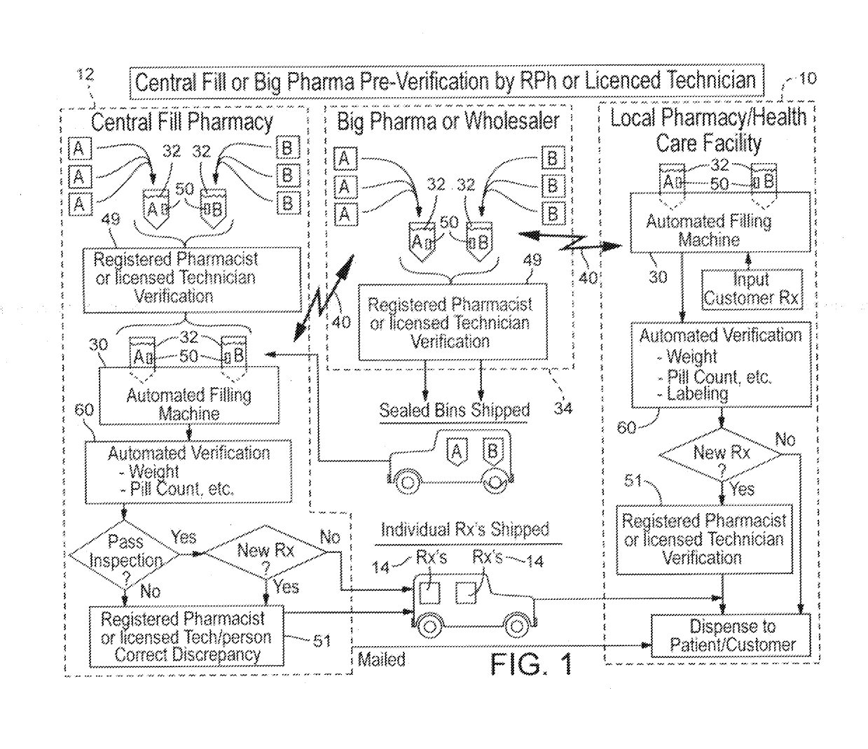

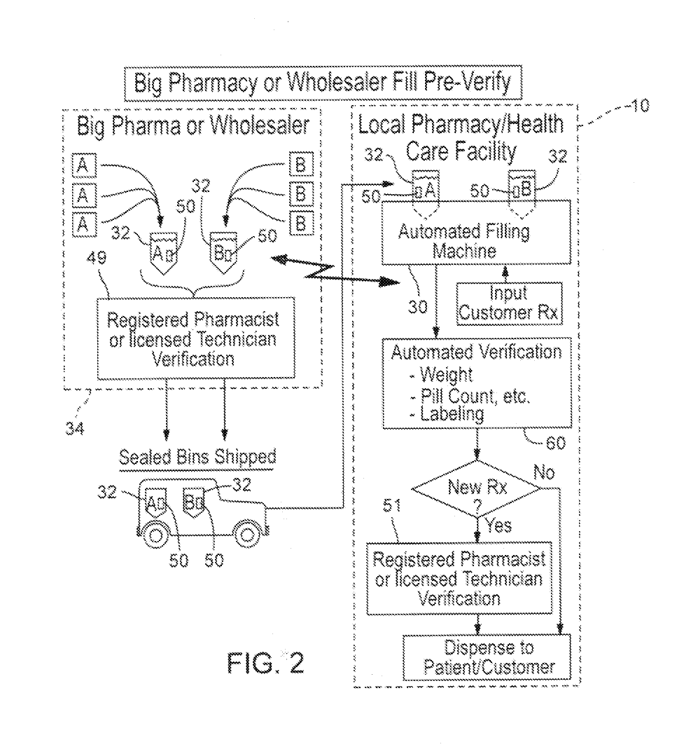

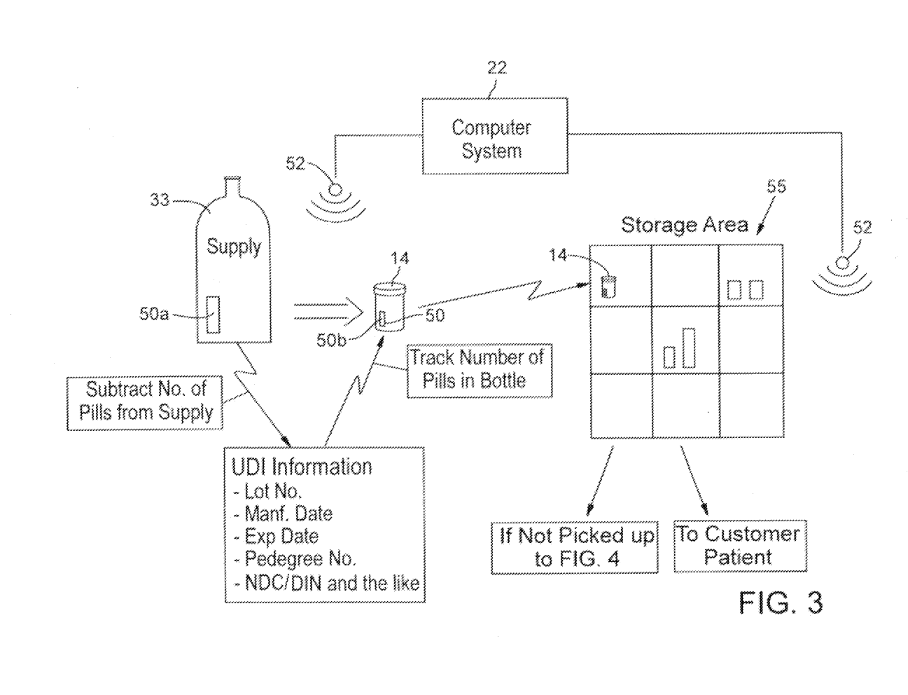

A pharmacy filling system is shown in FIGS 1-5. A local pharmacy 10 or healthcare facility may use a central fill pharmacy 12 to assist with filling an individual prescription order 14 as shown in FIG. 1, or it may fill the prescription order 14 within the local or retail pharmacy 10 itself as shown in FIG. 2. FIGS. 3-5 show an exemplar prescription medication dispensing system 20 to a customer or patent with a computer system 22 monitored and verified “return to stock” feature that minimizes medication loss while preserving the Unique Drug Identifier (“UDI”) of medication that is returned to stock. Each of these features is discussed in greater detail below.

Automated Prescription Filling with System Tracking and Verification

Regarding FIGS. 1 & 2, in both embodiments, an automated filling machine or system 30 is used to fill the prescription orders 14. The automated filling system 30 is located at the central fill facility 12 in the embodiment of FIG. 1, and at the local pharmacy 10 or healthcare facility in the embodiment in FIG. 2. At either location, the automated filling system 30 is stocked with medication supply bins 32 that have been filled and verified by a pharmacist and/or licensed pharmacy worker 49 at a remote location 34 such as a wholesaler or the medication manufacturer. The local pharmacy 10 or healthcare facility is preferably in electronic or computer communication 40 with the remote location 34 and central fill facility 12, if applicable.

The supply bins 32 are sealed and tagged at the remote location 34, preferably with an electronic tag 50 such as a barcode, RF tag, RFID tag, GPS tag, or the like, that travels with each supply bin 32. The tag 60 includes identifying information about the medication contained within the supply bin 32 to which it is attached. Preferably, the tag 50 is in communication with a computer system 22 that includes one or more tag readers 52 that detect the presence of the tag to determine its location. The computer system 22 includes a database that may include detailed information about the medication contained within the supply bin 32 including its location as well as UDI information about the medication.

More preferably, the tag 50 is a radio-frequency identification tag (“RFID”), and includes Unique Drug Identity (“UDI”) information regarding the medication contained within the bin 32. Such information can include the lot number, expiration date, drug name, drug strength, pedigree number, fill date, pharmacist/licensed pharmacy worker verification and identification, and the like for the medication contained in the bin.

The sealed bins 32 are transported to their respective automated filling system 30, which is either the central fill pharmacy 12 in the embodiment of FIG. 1, or the local pharmacy/health care facility 10 in the embodiment of FIG. 2. A local worker installs the bins 32 into the automated filling system 30 by breaking the seal. A local tracking system in communication with the computer system 22, such as a system disclosed in U.S. Pat. No. 8,479,988 to Louie, et al. or the like, uses a tag reader 52 or the like to automatically read the information contained on the tag 50 that is operably secured to the bin 32. The computer system 22 automatically verifies that the correct bin 32 has been placed in the correct location within the automated filling system 30 without requiring a local licensed pharmacy worker or pharmacists to verify the contents of the bin 32 or its proper location within the automated filling machine 30.

Should the computer system 22 detect a discrepancy, such as the wrong supply bin 32 being installed in the wrong location, the system 22 can alert a worker to correct the discrepancy or call of a licensed pharmacy worker or pharmacist to intervene to correct the situation.

During filling of the prescription order 14, either at the central fill pharmacy 12 in FIG. 1 or at the local pharmacy 10 or healthcare facility of FIG. 2, the supply bins 32 and the prescription orders 14 are tagged with electronic machine readable tags 50 that monitor and track their locations within the pharmacy and include information about the prescription orders and/or supply bin contents (e.g. UDI's) as needed.

The automated filling machine 30 may include standard automated filling verification systems 60 such as weight verification, label verification, pill count verification, video comparison of the pills to an image of the pill in a standard catalog of pills, and the like. These verification systems 80 verify that the automated filling machine 30 properly placed the correct medication and the correct amount of that medication into a container that has been properly labeled for a particular customer or patient. Should these systems detect a discrepancy, the system alerts a pharmacy worker of any discrepancies where that particular prescription order 14 is pulled from the system and manually inspected and corrected by a pharmacy worker before it is released to a customer or patient.

It can be appreciated, that with the foregoing systems, individual electronic tagging 50, tracking and monitoring of the supply bins 32 and the prescription orders 14, and automated verifications systems 60 downstream of the filling machines 30, a filled prescription order 14 for a refilled prescription may be dispensed to a customer with only one manual verification of the supply bins 32, by a healthcare worker, well upstream of the individual filling of the prescription 14. The downstream automated monitoring, testing, tracking and verification performed by the computer system 22 maintains the integrity of the supply bins 32 and the resulting filled prescription orders 14.

Of course, the filling system can include additional inspection stations along the filling path as needed to comply with local pharmacy dispensing regulations. For example, if a prescription order 14 is new, and calls for new prescription handling or patient counseling, a registered pharmacy worker and/or pharmacist 51 can be called in to verify that particular order and counsel the customer or patient. Refilled orders can pass this step saving licensed pharmacy worker's and pharmacist's time.

If desired, the pharmacist or licensed pharmacy worker 49 at a remote location 34 and/or the pharmacists or licensed pharmacy worker 51 at the local pharmacy 10 can each wear a tag 50 that is in communication with the computer system 22. This allows for the computer system 22 to monitor, track and document which workers performed which tasks related to both the supply bins 32 and individual prescription orders 14

System Tracking, and Verification of Unique Drug Identifier

Referring to FIGS. 3-5, the computer system 22 can monitor and track the Unique Drug Identifier (“UDI”) information about the medication in each supply container 33 within the system, such as lot number, manufacturer date, expiration date, drug name, drug strength, pedigree number, National Drug Code (“NDC”), Drug identification Number (“DIN”) and the like. The supply container 33 may be from a previously packaged bin 32 or a separate container that arrived at the pharmacy by other methods. As shown in FIG. 3, this UDI information can be transferred to each individual prescription order 14 when medication from that supply container 33 is transferred to an individual prescription order 14.

For example, the supply container can include a first machine readable tag 60a that is readable by a tag reader 52 in communication with a computer system 22. A second machine readable tag 50b can be operably secured to the individual prescription order 14. The first tag 50a can include UDI information about the medication contained in the supply container 33 including the remaining volume or number of pills contained therein. When the individual prescription order 14 is filled and a portion of the contents of the supply container 33 are transferred to the container containing the individual prescription order 14, the computer system 22 can detect this activity and associate the individual prescription order 14 of that tagged individual prescription container to a customer or patent's prescription order. The computer system can transfer the resulting UDI information of the supply container 33 to the tag 50b associated with the individual prescription order 14. This UDI information from the supply container 33 travels with the individual prescription order 14 to a storage area 55 until that order is dispensed to a customer or patient.

UDI information Preserved When Medication is Returned to Stock

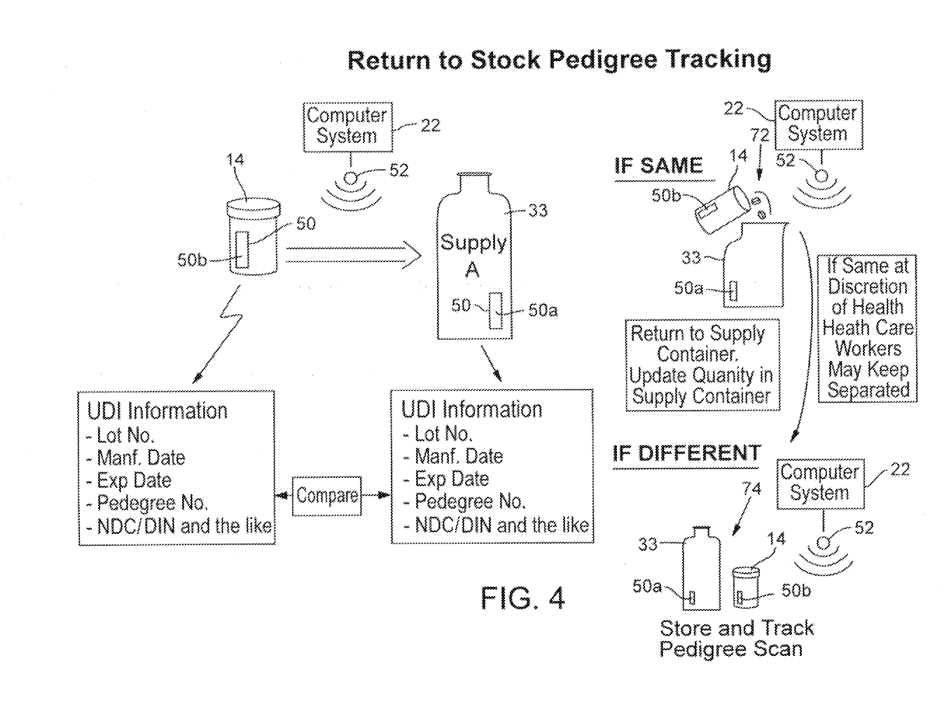

Referring to FIG. 4, should an individual prescription order 14 be returned to stock or the like, the system can track and store the UDI information about the returned medication, thereby allowing it to fee re-dispensed without risk of if becoming expired or without knowing its exact pedigree. The computer system 22 can read the tag 50b associated with the returned prescription order 14 and the tag 50a associated with the supply container 33 and compare the UDI's of each, if key items of UDI's are identical, such as lot number, expiration date, NDC and pedigree number, the returned medication can be placed back into the supply container 33 without compromising the integrity of the supply container 33 as shown by arrow 72 in FIG. 4. The system can activate one or more transducers if the transfer of the returned medication is not authorized and alert a pharmacy worker if the UDI of the supply container 33 has been compromised.

Alternatively, if the UDI's between the returned medication and the supply container 33 do not match, the returned medication can remain within the storage area for use to fill a new prescription order as shown by arrow 74 in FIG. 4. The computer system 22 can alert a pharmacy worker whether to use medication from the returned prescription order or from the supply container.

Having described how the computer system 22 maintains and tracks UDI information from the supply containers 33 to the individual filled prescription orders 14. It can be appreciated that medication loss can be minimized by preserving UDI integrity of the returned to stock medications, and inadvertent dispensing of expired medications to customers and patents can be eliminated.

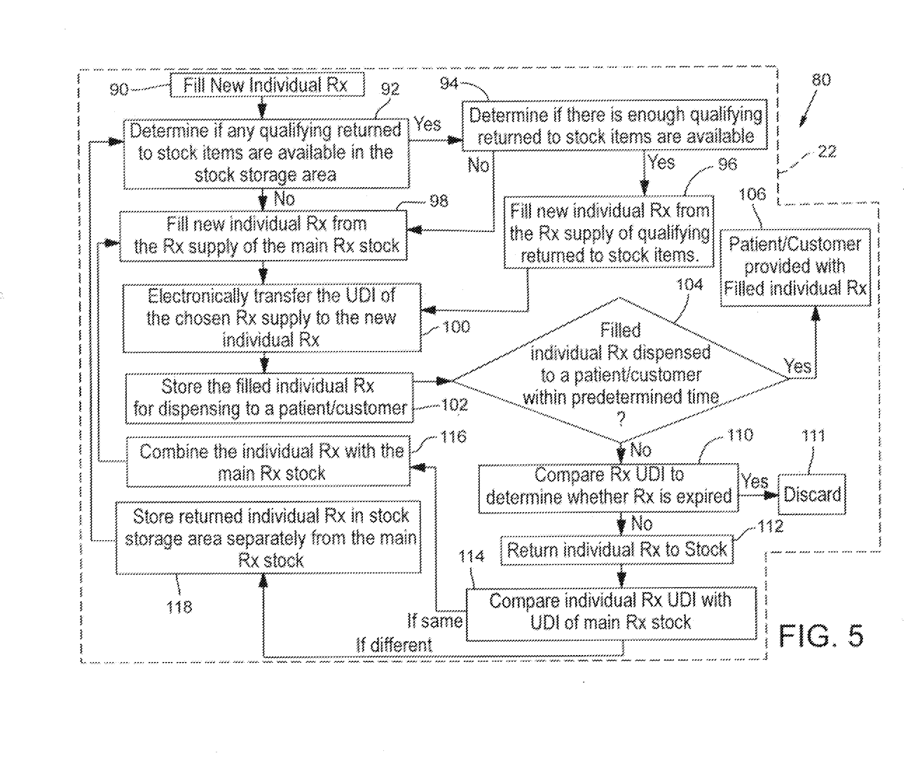

An exemplar individual prescription filling system 80 taking full advantage of maintaining medication UDI information integrity throughout the filling process is shown in FIG. 5. In step 90, a new individual prescription order is provided to the pharmacy. The pharmacy first determines if there are qualifying returned to stock medications available to fill the new prescription order (Step 92). If there is, the system next determines if there is enough of the returned to stock medication to fill the new prescription order (Step 94) and if there is the new prescription order is filled with from the returned to stock supply (Step 96). In cases where the supply of returned medication is greater than the amount dispensed to the new individual prescription order the computer system may update the volume or quantify of medication in the returned to stock supply as medication is dispensed to fill the new prescription order.

Alternatively, if there is none or not a sufficient amount of returned to stock medication to fill the new prescription order, the system directs a pharmacy worker or an automated fill system to fill the new prescription order from the main stock supply container of the prescribed medication (Step 98).

After the individual filled prescription order is filled either from an existing returned to stock supply or from the main stock supply, the computer system transfers the UDI information of the source supply to the computer readable tag associated with the individual prescription order (Step 100). More preferably, the computer system also monitors and tracks the volume or pill count in both the supply container and the volume or number of pills placed in the container of the individual prescription. For example, it consults the database associated with that customer/patient and determines the number of pills prescribed and deducts that amount from the selected supply container and adds them to the filled individual prescription order.

The filled individual prescription order is then stored for dispensing to a customer or patient (Step 102), and a clock is initiated to track how long the individual prescription order remains in the storage area. Preferably, the individual prescription is stored in an area that is in communication with the computer system 22 to automatically monitor, detect, and log user access.

As shown in Step 104, if the individual prescription order is dispensed to a patient or customer within a predetermined time, further tracking of the UDI information and location tracking of the customer's order can stop (step 108). The UDI and other information can be stored in a database for further reference or documentation as needed.

Alternatively, if the individual medication is not dispensed to a patient or customer OR within a predetermined time, the medication within the individual prescription can be returned to stock (step 112). However, because the UDI information of the supply container is preserved and transferred to each individual prescription filled, should a medication contained within a filled prescription order waiting for pick-up expire before it is picked-up by a customer or dispensed to a patient (step 110), the system can flag the discrepancy to a pharmacy worker, such as by activating a transducer on the tag associated with that prescription order, who can correct the situation before the customer seeks to pick up the medication or before it is dispensed to a patient (step 111). The computer system 22 can further prevent dispensing of an expired medication to a patient or customer by taking additional security steps such as calling for a pharmacist override before it will unlock or locate the prescription order for the pharmacy worker, or by activating an audible warning alarm or the like.

If desired, the computer system can also update the UDI information of the individual prescription order 14 and the supply container 33 with a user entered or pharmacy pre-selected configurable date. Preferably, this configurable date is earlier than the expiration date of the medication contained in the respective individual prescription order 14 and supply container 33, and it is selected to give a patient or customer a reasonable time to use the medication before it will expire. The previously described flagging and warning system for expired medications could also be activated upon reaching this configurable date, thereby preventing the dispensing of medications that do not have a reasonable time to be used before they expire.

Also, the electronic tracking of pedigree information of medications in the supply container also allows the pharmacy to dispense medications that are closer to their expiration dates first, and avoid dispensing medications that are too close to their expiration dates to be meaningfully used by a customer or patient before they expire. Using the oldest inventory first and avoiding disposing of expired or nearly expired medications with this system also saves money for the pharmacy.

The computer system first compares the UDI information of each individual prescription order with the UDI information of the supply container for that particular medication (step 114). If the UDI information is the same, the medication contained within the individual supply container can be returned to the supply container (step 118). The pill count or volume of medication in the individual prescription order returned to the supply container can be automatically added by the computer system to the UDI information of the supply container. Alternatively, if the UDI information between the individual prescription order differs from the UDI information of the supply container, the returned to stock medication can be stored and tracked for further use (step 118) as previously described, Of course, the worker may choose to store separately even if the UDI's match to save time.

One skilled in the relevant art will recognize that numerous variations and modifications may be made to the configurations described above. For example, if desired in step 94, if there is not a sufficient amount of medication in a returned to stock supply of medication, a pharmacy may fill only a partial amount of an individual prescription order from the returned to stock supply. If it does this, it can pull the remaining supply from the main stock of the medication supply, if it combines medication from these two sources of supply medication into one individual container, the UDI information of that container is compromised. Accordingly, the computer system will track this individual prescription order to ensure that it is not returned to stock for reuse. Alternatively, the pharmacy may provide two individual containers, each with an individual machine readable tag and each containing the UDI information of the supply source from which it was filled. In such case, the medication contained within each individual container may be returned to stock as previously described while maintaining UDI integrity of all the medications.

Also, the storage area 55 (FIG. 3) for filled prescription orders 14 needs to be spaced apart from the storage area of the supply containers, in such case, should a filled individual prescription order be flagged for being returned to stock and stored in the same area where the supply of that medication is also stored, the “return to stock” function can be purely electronic, whereby the computer system simply reclassifies that that individual prescription order as being returned to stock medication without that item actually being moved. Of course, any customer identifying labeling information would still need to be removed before the returned item could be dispensed to a new customer.

Accordingly, the foregoing description of embodiments has been provided for purposes of illustration and description. It is not intended to be exhaustive or to limit the disclosure. Individual elements or features of a particular embodiment are generally not limited to that particular embodiment but whom applicable, are interchangeable and can be used in a selected embodiment, even if not specifically shown or described. The same may also be varied in many ways. Such variations are not to be regarded as a departure from the disclosure and all such modification are intended to be included within the scope of the disclosure.

We claim:

1. A pharmacy prescription order filing system comprising:
a stock supply container received within a pharmacy: the stock supply container containing a stock supply of medication, the stock supply container having a first machine readable tag operably secured thereto;
an individual prescription order container having a second machine readable tags operably secured thereto;
a tag reader in communication with a computer system for detecting the presence of and distinguishing the first and second tags from each other;
a database in communication with the computer system containing at least one Unique Drug identifier (“UDI”) of the stock supply of medication contained in the stock supply container; and,
the computer system detecting the second machine readable tag within the proximate location of the first machine readable tag when a portion of the stock supply of medication is dispensed from the stock supply container to the individual prescription order container and automatically associating the at least one unique drug identifier with dispensed medication placed into the individual prescription order container.

2. The pharmacy prescription order filling system of claim 1, wherein the prescription order container containing the dispensed medication is moved to a storage area for distribution to a customer or patient.

3. The pharmacy prescription order filling system of claim 2, wherein the computer system monitors the amount of time the prescription order remains in the storage area and alerts a pharmacy worker to return the prescription order to stock storage after a predetermined time.

4. The pharmacy prescription order filling system of claim 3, wherein the computer system compares the unique drug identifier of the dispensed medication with the unique drug identified of the stock, supply container and authorizes returning the dispensed medication to the stock supply container if the two unique drug identifiers match.

5. The pharmacy prescription order filling system of claim 3, where the computer system compares the unique drug identifier of the dispensed medication with the unique drug identifier of the stock supply container and tracks the location and availability of the dispensed medication for future dispensing while spaced apart from the supply container when the two unique drug identifiers do not match.

8. The pharmacy prescription order filling system of claim 1 wherein the computer system allows a configurable date to be added to the unique drug identifier, and the computer system alerts a pharmacy worker if the individual prescription order is past the configurable date.

7. The pharmacy prescription order filling system of claim 6, wherein the computer system prevents the dispensing of the prescription order to a customer or patient past the configurable date.

8. The pharmacy prescription order filling system of claim 8, wherein in the configurable date is earlier than the expiration date of the UDI for the medication contained in the supply container from which the individual prescriptions medication was drawn.

9. The pharmacy prescription order filling system of claim 8, wherein the computer system prevents the dispensing of the prescription order to a customer or patient past the expiration date of the UDI for the medication contained in the supply container from which the individual prescription's medication was drawn.

10. The pharmacy prescription order filling system of claim 1, wherein the first and second machine readable tags are a radio frequency identification (“RFID”) tags in communication with the computer system.

11. The pharmacy prescription order filing system of claim 1, further including a thud machine readable tag in communication with the computer system to monitor the location and performance of the pharmacy worker relative to the supply container and the individual prescription order.

12. The pharmacy prescription order filling system of claim 1, where the stock supply container containing a stock supply of medication is filled at a remote location and transported to the pharmacy.

13. The pharmacy prescription order filling system of claim 12, further including a second tag reader in communication with the computer system for detecting the presence and location of the first fag at the remote location.

14. The pharmacy prescription order filing system of claim 1, wherein the database containing the UDI is received within memory spaced apart from the first and second fags.

15. The pharmacy prescription order filing system of claim 1, wherein the database is stored in memory operably received with the first and second tags.

18. The pharmacy prescription order filling system of claim 1, wherein the medication UDI information is selected from the group consisting of drug name, drug strength, lot number, manufacture date, expiration date, pedigree number, and National Drug Code.

17. The pharmacy prescription order filling system of claim 1, further including an automated filling machine operably received within the pharmacy, and the stock supply container operably received within the automated filling machine.

18. The pharmacy prescription order filling system of claim 1, wherein the computer system monitors the storage area containing the prescription order for automatic monitoring, detection and logging of user access.
